SEQUENCE LISTING (2 sequences)

SEQ ID NO: 1 - DNA, 18 bp, Artificial Sequence
  DNA probe
tgacggaggttgtgaggc

SEQ ID NO: 2 - DNA, 18 bp, Artificial Sequence
  DNA probe
gcctcacaacctccgtca

(19) United States
(12) Patent Application Publication
(10) Pub. No.: US 2004/0110278 A1
(43) Pub. Date: Jun. 10, 2004

(54) Solution mixing method and apparatus

(75) Inventors: Kazunori Okano, Shiki (JP); Kunio Harada, Hachioji (JP); Yoshinobu Kohara, Kokubunji (JP); Hideyuki Noda, Kokubunji (JP)

Correspondence Address: Stanley P. Fisher, Reed Smith LLP, Suite 1400, 3110 Fairview Park Drive, Falls Church, VA, 22042-4503, US

(73) Assignee: Hitachi., Ltd.

(21) Appl. No.: 10/684,473

(22) Filed: Oct. 15, 2003

(30) Foreign Application Priority Data
Oct. 22, 2002 (JP) 2002-307535

(51) Int. Cl. C12M 1/34

(52) U.S. Cl. 435/287.2; 435/289.1

(57) ABSTRACT

The invention provides a solution mixing method by moving relatively at least one DNA array to which probes are fixed to a moving plate with a solution entertained therebetween, while the solution contains a substance of interest which is intended to react with the probes. Such a method can be implemented by an apparatus including a substrate holding plate with at least on substrate and a moving plate (moving along a straight line or revolving), an apparatus including a spinning compartment for spinning about a central axis and a holding plate for holding probes on its surface when being spun along the spinning compartment.

### BACKGROUND OF THE INVENTION

1. Field of the Invention

This invention relates to a solution mixing apparatus, especially for efficiently reacting substances of interest with probes immobilized on an arrays.

2. Description of Related Arts

DNA microarrays or Gene arrays have been widely used for analyzing DNAs or for monitoring the gene expression on a array thereby identifying genetic variations associated with disease to discover new drug targets. The array, which consists of a small glass plate encased in plastic, is manufactured using a process similar to the one used to make semiconductor microarrays. On the surface, hundreds of thousands of oligonucleotide probes are immobilized and arranged spottily at extremely high densities. These probes are synthetic single or double stranded DNAs whose sequences are identical to a normal gene.

The probe-substance of interest combination is not only for DNA. They may be (1) a nucleic acid—a single-stranded nucleic acid, (2) a nucleic acid—a double-stranded nucleic acid, (3) an antigen—an antibody, (4) an antibody—an antigen, (5) a ligand—a receptor, (6) a receptor—a ligand, (7) a substrate—an enzyme, (8) an enzyme—a substrate, (9) peptidyl nucleic acid—a nucleic acid, or (10) a nucleic acid—peptidyl acid, respectively.

A plurality of probes or a sample of a nucleic acid, protein, tissue section or like are immobilized on an array substrate, then the sample-probe reaction occurs, and whether there is a change in the level of a gene expression or protein expression in the sample is analyzed. Generally, the sample-probe reaction, for example, hybridization reactions are carried out by dropping a hybridization solution containing the sample onto a DNA array substrate with probes immobilized thereon, covering the substrate with a cover glass such that the hybridization solution may not evaporate, placing the substrate in a wet box or a tightly closed cassette, and maintaining the substrate at a constant temperature for a fairly long period of time (over 12 hours).

It is well known that shaking and/or stirring of the hybridization solution to cut down the hybridization time and to conduct the hybridization homogeneously. Currently, a rotisserie type rotating device for the hybridization with membranes, or a shaking platform for oscillating the hybridization solution with an array substrate are available for use.

U.S. patent application Ser. No. 2001/0,046,702 describes an array hybridization device having a substantially planar bottom 34, a cover 35, at least one fluid port 36 and at least one adjustable spacing element 37 for adjusting the spacing between an array and the bottom surface as shown in FIG. 15. In using the subject devices, an array is placed on the at least one adjustable spacing element in the chamber 39 and the space between the array and the bottom surface is adjusted by moving the at least one adjustable spacing element 37. The adjusted array is contacted with at least one biological sample introduced into the chamber 39. A mixing means, such as a resistor, an ultrasonic element, a recirculation pump, a roller, an adjusting spacing element, or a solenoid, is provided to mix the solution.

There is a method for agitating solution contained between two substrates by introducing bobbles into the space therebetween described by the U.S. Pat. No. 6,186,659. The bobbles are produced with a discrete heat source, such as a resistor, and moved in the solution by creating a temperature gradient thereby mixing the solution.

By the method of trickling the hybridization solution containing the conventional sample on the array board to which the probe was fixed, the reaction time of hybridization is the prolonged as long as 12 hours, and the reaction efficiency is slow. The above-mentioned patents did provide more efficient methods than the set still method in FIG. 15. However, their reactions are provided inhomogeneous for different spots on one probe-attached area. In addition, the resulted reaction efficiency is still not satisfactory.

Currently, there is a demand for a solution mixing method and apparatus for providing efficient and homogeneous probe-substance reactions.

### SUMMARY OF THE INVENTION

It is a purpose of this invention to provide a method and an apparatus for improving solution mixing efficiency and uniformity.

It is another purpose of this invention to provide such a method and an apparatus which are easy and economical to operate as well as configured with a simple structure.

The above-mentioned purposes are achieved by moving relatively the holding plate holding the array to which probes are fixed to a moving plate with a solution entertained therebetween. It includes various situation: (1) the holding plate moves and the moving plate stays, or (2) the moving plate moves and the holding plate stays, or (3) both of the holding plate and the moving plate move. The solution contains at least one substance of interest which is intended to react with the probes. Such a method can be implemented by an apparatus including a substrate holding plate with at least one substrate and a moving plate (moving along a straight line or revolving), an apparatus including a spinning compartment for spinning about a central axis and a holding plate for holding the substrate when being spun along the spinning compartment.

The flow volume of the solution are almost the same in every location on probe-attached area on the substrate such that the reaction homogeneity improves significantly. Therefore, in addition to high reaction efficiency, a reaction progresses uniformly at different locations of probe-attached area.

Here, any of nucleic acid, -single stranded nucleic acid, -double stranded nucleic acid, antigen-antibody, and antibody-antigen, ligand-receptor, and receptor-ligand, substance-enzyme, and enzyme-substance, peptidyl nucleic acid-nucleic acid, or nucleic acid-peptidyl nucleic acid are sufficient as the relation between an array and sample solution as mentioned above. Since churning efficiency poses a problem and high reaction efficiency is especially acquired by considering as the above-mentioned composition in small DNA and small RNA of a molecular weight, there is especially an effect.

The plates and the probe-attached area(s) of the embodiments take a variety of configurations, with the only limitations being (1) that the holding plate is bigger enough to cover the moving plate during its movement, and (2) that the moving plate is bigger enough to cover the probe-attached area during its movement. The configurations in conjunction with limiting the amplitude of the relative movement ensures the edge of the moving plate do not move over the top of the edge of the probe-attached area. As such, there are always a safe margin therebetween such that no spillage of the reaction solution form the edges.

Furthermore, the above-mentioned purposes are achieved by generating centrifugal force by rotating relatively placing component which holds the substrate and the solution inside to the substrate. Such a method can be implemented by an apparatus including the placing component holding the substrate inside, an apparatus including a spinning compartment for spinning the placing component.

The centrifugal force generated by the rotaition of the placing component force the substrate to be set at internal surface of the placing component where the backside of the probe-attached area in the substarate is faced to the internal surface of the placing component. The centrifugal force also force the solution to be supplied onto the substrate cyclically and uniformly. Therefore, in addition to high reaction efficiency, a reaction progresses uniformly at different locations of probe-attached area.

### BRIEF DESCRIPTION OF THE DRAWINGS

The foregoing and additional features and characteristics of the present invention will become more apparent from the following detailed description considered with reference to the accompanying drawings, in which like reference numerals designate like elements and wherein:

FIG. 1 shows a solution mixing apparatus of a first embodiment according to the invention;

FIG. 2. Is a partial cross-sectional view of the first embodiment in FIG. 1;

FIG. 3 shows how an injector and a sucking means work according to the invention;

FIG. 4 shows a second way to inject the chemical solution according to the invention;

FIG. 5 depicts the relation between the fluorescence intensity and the reaction time according to the invention and conventional art;

FIG. 6 depicts the relation between the fluorescence intensity and the distance between the holding plate and the moving plate according to the invention;

FIG. 7 shows a pair of spinning shafts and a pair of auxiliary plates and a base plate on the moving;

FIGS. 8A-8D show the direction and the relative moving vectors of the moving plate upon different positions of the probe-attached areas during one revolving cycle according to the invention;

FIGS. 9A-9D is a perspective view of a pair of spinning shafts and a pair of auxiliary plates and a base plate on the moving plate of the second embodiment in FIG. 7;

FIG. 10 show a first variation of the first embodiment;

FIG. 11 show a second variation of the first embodiment;

FIGS. 12A-12C show a solution mixing apparatus of a second embodiment according to the invention;

FIG. 13. Is a partial cross-sectional view of the second embodiment in FIG. 12;

FIG. 14 shows a solution mixing apparatus for of a third embodiment according to the invention;

FIG. 15A is a top view of a flexible holding plate of the third embodiment, and FIG. 15B is a cross-sectional view of a spinning compartment of the third embodiment; and

FIG. 16 shows a prior art of solution mixing apparatus.

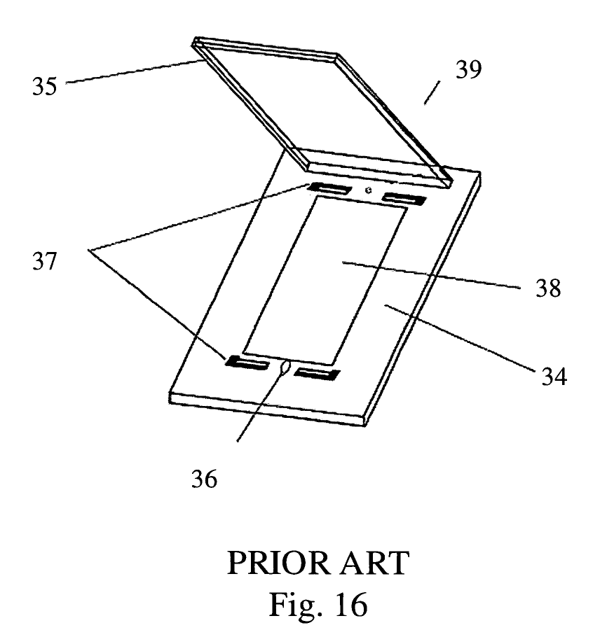

### DESCRIPTION OF THE PREFERRED EMBODIMENTS

In the following, several embodiments of the present invention are described in detail referring to the drawings.

### Embodiment 1

The solution mixing apparatus therein of the first embodiment of the invention includes: (1) a holding plate for holding the substrate; and (2) a moving plate with one surface being placed close to the surface of the substrate so as to accommodate a chemical solution there-between with a gap from the surface of the substrate, said moving plate moves relatively to the holding plate to mix the chemicals with the chemical solution.

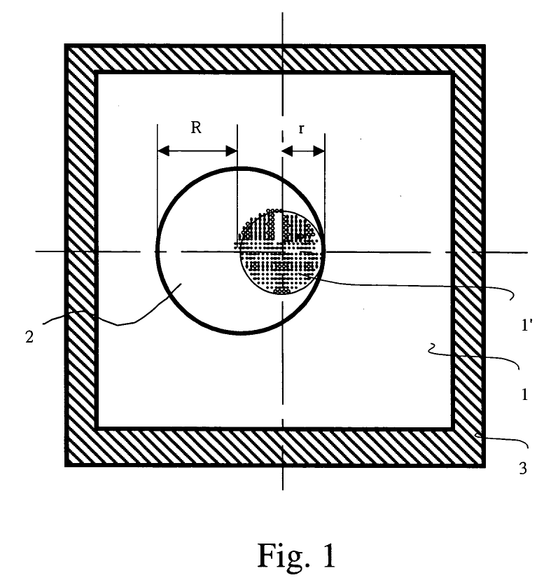

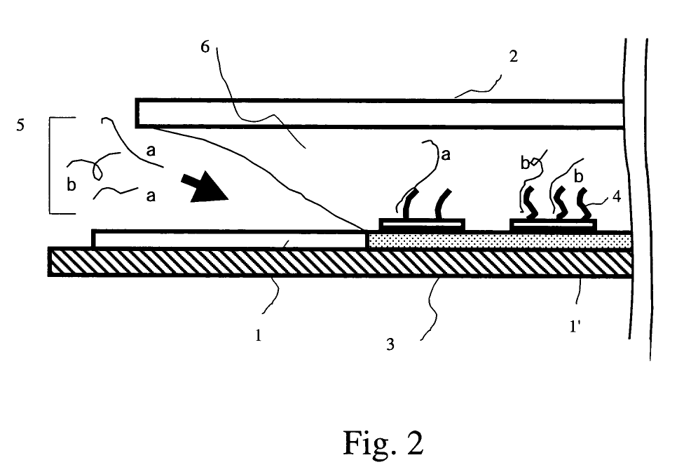

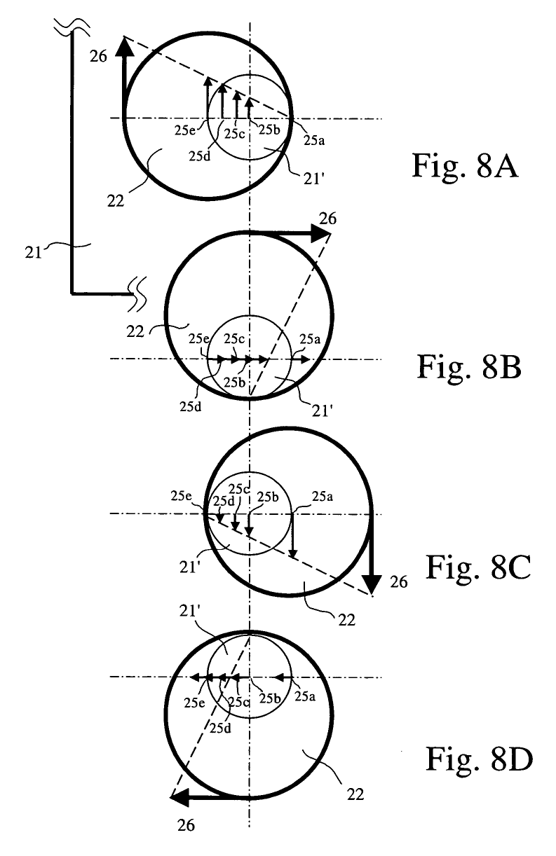

Rather than having the substrate and the chemical solution moving together as in the conventional shaking platform, the first embodiment of the invention has a holding plate 3 for holding a substrate 1 and a moving plate 2 as shown in FIG. 1. The holding plate 3 and the moving plate 2 move relatively to each other as shown in FIGS. 8A-8D to mix chemicals held between the surfaces (i.e., at least one substance of interest 5) of the substrate 1 with a chemical solution 6 accommodated therebetween with a gap from the chemicals as shown in FIG. 2. In particular, a probe-attached area 1′ are on the substrate right at its center, and the moving plate 2 revolves about the probe-attached area 1′ like a hulahoop. The probe-attached area 1′ is attached with plural kind of probes 4 each of which captures a plurality of the substance of interest 5 during the revolving movement. As shown in FIG. 2, two kinds of substances of interest 5a, 5b are attached to the probe-attached areas 1′ respectively. Therefore, more than one kind of reactions can be conducted in the same space. (The number of the probe-attached areas is selected as two to simplify the discussion.)

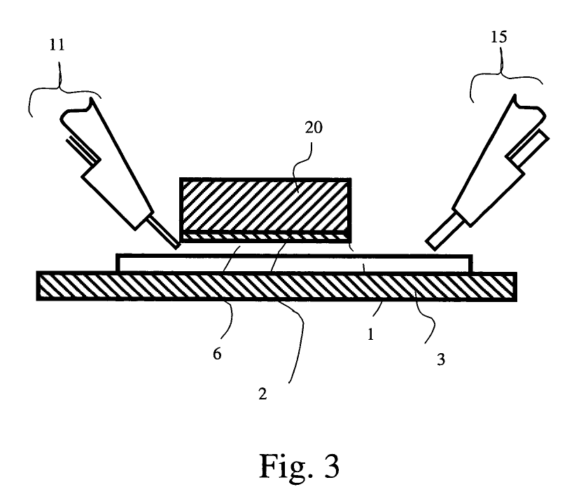

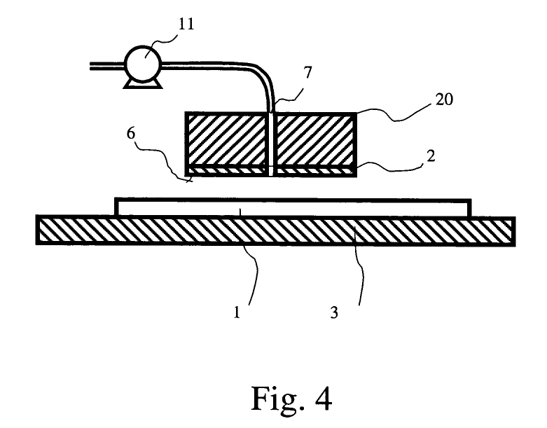

The chemical solution 6 can be injected into the space between the susbstrate 1 and the moving plate 2 with an solution injector 11 form the edge (FIG. 3) or via a channel 7 connecting through the center of the moving plate 2 to the space (FIG. 4). The edge injunction method is preferred since it takes a shorter guiding route to the space thereby reducing the dead solution wasted in the guiding port 12. The surface of the moving plate 2 is hydrophilic such that the chemical solution 6 is introduced into the space by capillary phenomenon. The remaining solution is sucked out of the space after the chemical reaction or the hybridization with a sucking means 15.

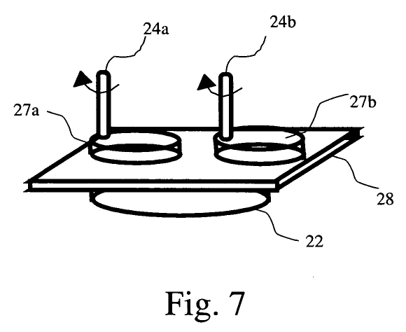

In addition to the holding plate 3 and the revolving plate 2, the solution mixing apparatus further comprises a moving mean which includes a base plate, at least one shaft, and at least one auxiliary plate. The base plate has the moving plate fixed on one side and the auxiliary plate rotatably attached on another side, while the auxiliary plate has the base plate rotatably attached on one side and the shaft fixed on another side. In a preferred embodiment as shown in FIG. 7, the moving means includes a base plate 28, a pair of the shafts 24a, 24b, a pair of the auxiliary plates 27a, 27b. In FIG. 7, the moving plate 22 and the auxiliary plates 27a, 27b are circular, and the base plate 28 is rectangular, and the moving plate is fixed right under a center of the base plate on one side of the base plate. One shaft 24a is fixed at a distance from a center of the auxiliary plate 27a while another shaft 24b is fixed at the same distance from a center of another auxiliary plate 27b.

Each of the auxiliary plates 27a, 27b has its spinning axis rotably fixed on a base late 28, which in turn fully fixed with the moving plate 22. Each of spinning shafts only spins but does not move or revolve relatively to the earth, the holding plate 3, or the probe-attached area 1′. As the spinning shafts 24a, 24b spin, the respective auxiliary revolving plates 27a, 27b revolve and in turn revolve the base plate 28 such that the revolves moving plate 22 revolves along. Accordingly, the moving plate 22 orbits the center of the holding plate 3 (also the center of the probe-attached area 1′) without any spinning to its own center like the earth revolves about the sun but without any self-spinning. In other words, the edge of the moving plate 2 whirls like a hulahoop around the circumference of the probe-attached area 1′ (like a human body) on top of the holding plate 3. However, in-this case, the probe-attached area 1′ (body without any hip movement) does not move but only the moving plate 2 moves (only the hulahoop moves).

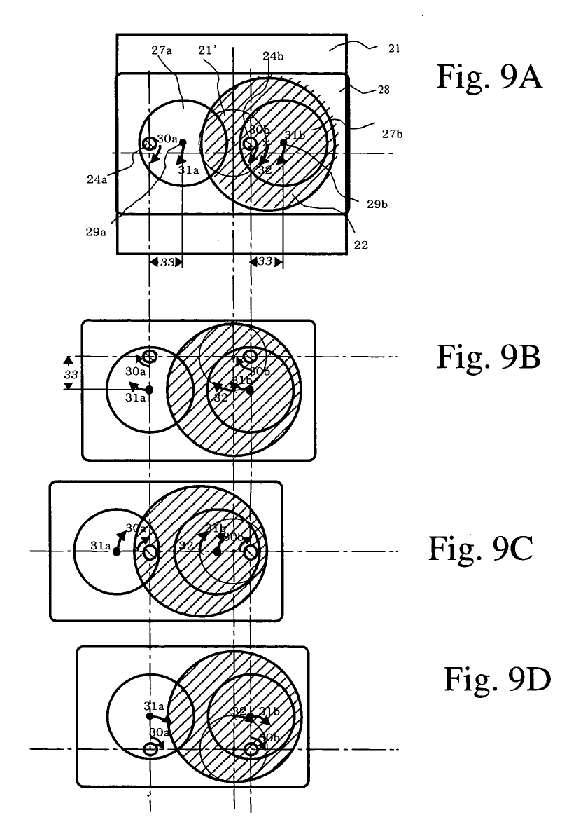

FIGS. 9A-9D is a perspective view of a pair of spinning shafts and a pair of auxiliary plates and a base plate on the moving plate of the second embodiment in FIG. 7. The auxiliary plates 27a, 27b has the base plate 28 rotatably attached on one side via pivots 29a, 29b. The auxiliary plates 27a, 27b is fixed to the shafts 29a, 29b. A central axis of the revolving of the auxiliary plate is a center of the pivots and the distance between them is 33 shown in FIG. 9A. Thus, the moving plate 22 is revolving with shifting its center axis of the revolving with the same distance as 33 shown in FIG. 9A. For example, when a radias of the moving plate 22, the probe-attached area, and the auxiliary plate is 40 mm, 20 mm, and 13 mm respectively, the distance between the central axis of the revolving of the auxiliary plate and the center of the pivots is about 10 mm. In perspective view, the moving plate covers the whole of the probe-attached area 21′. Also, the moving plate 22 revolves onto the probe-attached area 21′ while overlapping their edge.

In FIG. 8A, the moving vectors created by the motion of the moving plate 22 relative to the probe-attached area toward the 12 o'clock position increases proportionally from the positions 25a (0), 25b, 25c, 25d, to 25e (a maximum value). In FIG. 8B, all the positions 25a-25e experience the moving vectors created by the moving plate 2 toward the 3 o'clock position. In FIG. 8C, the moving vectors toward the 6 o'clock position increases from 0 at the position 25e, then the positions 25d, 25c, 25b, to a maximum value at the position 25a. In FIG. 8D, all the positions 25a-25e experience the moving vectors toward the 9 o'clock position. Although the magnitude of the moving vectors are experienced differently on each point/location of the substrate at each time point, the sum of the vectors after one cycle is zero, i.e. equal on each point/location of the substrate. It means the volume of the flowing solution on each point/location of the probe-attached area is the same per revolving cycle. Accordingly, each of the positions 25a-25e experiences the same volume of solution flow (regardless of directions of the moving vectors) during each revolving cycle. 25a-25e show the moving vectors upon the positions 25a-25e during one revolving cycle and their sum is 0 per cycle. The revolving speed of the revolving plate 2 is 0.1-60 seconds per cycle, preferably 0.9-30 seconds per cycle.

Generally, the surfaces of the moving plate, the holding plate, and the substrate are substantially flat and smooth. However, for optimization of a reaction, grooves or canals may be on the surface of the moving plate to catch the air bubbles incurred by high temperature maintained between the plates. Regarding detecting the incurred reaction efficiency, it can be carried out by adding labels, such as a fluorescent substance, to the substance of interest (to be combined with the probes), before the reaction. The fluorescence is then detected after the reaction so as to analyze the existence and quantity of the substance of interest. This detection may carried out by an external equipment (not shown). Alternatively, such equipment can be incorporated with the system of the invention.

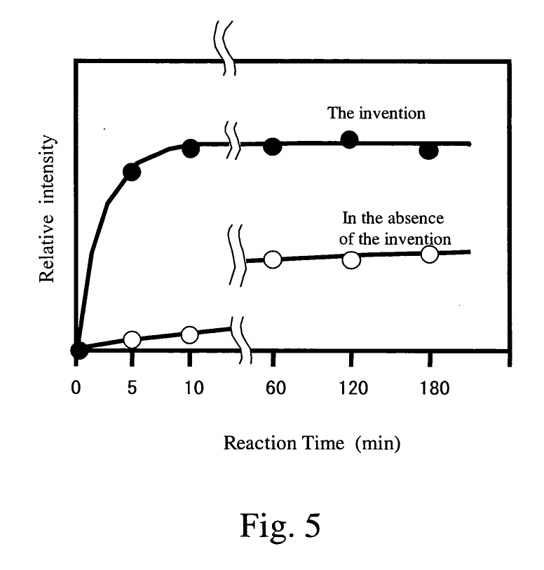

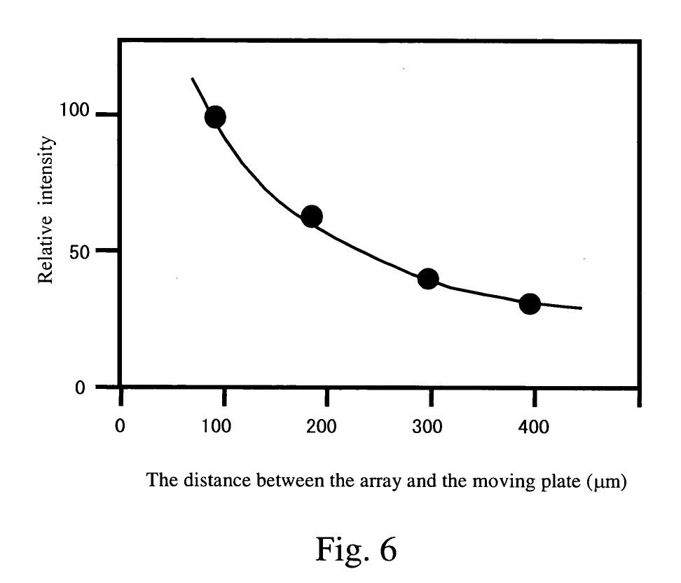

FIG. 5 and 6 show the fluorescence intensity emitted from the first embodiment and detected by another apparatus(not shown). In FIG. 5, the ordinate denotes the fluorescence intensity (instrument units), and the abscissa denoted the reaction time (minute) of the sample solution spotted. The fluorescence intensity after the hybridization reaction is detected at each indicated time with a plurality of independent sets. The measurements may be taken by draining out the solution of one of the independent sets at each measuring time point to measure directly above the probe-attached areas. Sufficient fluorescence intensity was obtained in reaction time in about 10 minutes according to the first embodiment. The invention significantly accelerate the reaction speed such that the reaction is almost saturated after the first hour (in block dots). On the other hand, the set-still situation with dropping the hybridization solution onto the substrate reached only half of the fluorescence intensity after 180 minutes. It took over 12 hours for the reaction to become saturated in the absence of the invention (in white dots). The experiment conditions follows the description in Nucleic Acids Research e87, No. 16, Volume 30, 2002. The sample DNA used here is 0.1 nM synthetic oligonucleotide (Sequence number 1) that has eighteen bases and 5-′end labeled with sulforhodamine 101. The probe fixed onto the probe-attached area is the complementary sequence (Sequence number 2) to the synthetic oligonucleotide and has 5′-end with SHgroup. After introducing amino group onto the surface of the glass substrate by treated with 3-amino-propyl-trimethoxycsilane, the amino group on the glass surface and the SH group of the probe are bridged with N-(11-maleimidoundecanoxyloxy) succinimide to fix the probe onto the substrate.

In FIG. 6, the ordinate denotes the fluorescence intensity (instrument units), and the abscissa denoted the distance (μm) between the substrate held on the holding plate 1 and the moving plate 2. The reaction efficiency increases along with the decrease of the distance, i.e., the plates getting closer in the range of 50 to 400 μm. In particular, when the distance between the plates is 50 to 100 μm, the reaction increases exponentially along with the decrease of the distance, between the plates. For example, a substance of interest comprising 10-base long of double-strained DNAs such that it has a length of 24 nm, and a length of each probe is about 10-100 nm. The length of the probe-substance combination is in the range of 100s nm, which is ignorable comparing with the minimum distance between the plates is 50,000-100,000 nm. As such, the gap between the probes on the substrate and the moving plate is approximate the same as the distance between the moving plate 2 and the substrate 1.

Furthermore, the reaction efficiency varies depending on different locations on the probe-attached area when the reaction is done with set-still situation with dropping the hybridization solution onto the substrate. The relative standard deviation of the fluorescence intensity of the spots in the probe-attached area drops to 1/13 by applying the solution mixing method according to the present invention. In other words, the invention significantly improve the homogeneity of hybridization.

The apparatus may further comprise a temperature regulation device mounted on the moving plate to maintain the temperature within the hybridization space at an optimized level or levels according to pre-selected temperature profiles. As shown in FIGS. 2 and 3, the themparature regulation device 20 may be installed on the top of the moving plate 2 or incorporated with a stand-like component to be installed below the substrate 1. Furthermore, it may be installed externally to control the temperature of the system via the space surrounding the system. The combination of these installation methods with the revolving motion improves the reaction efficiency.

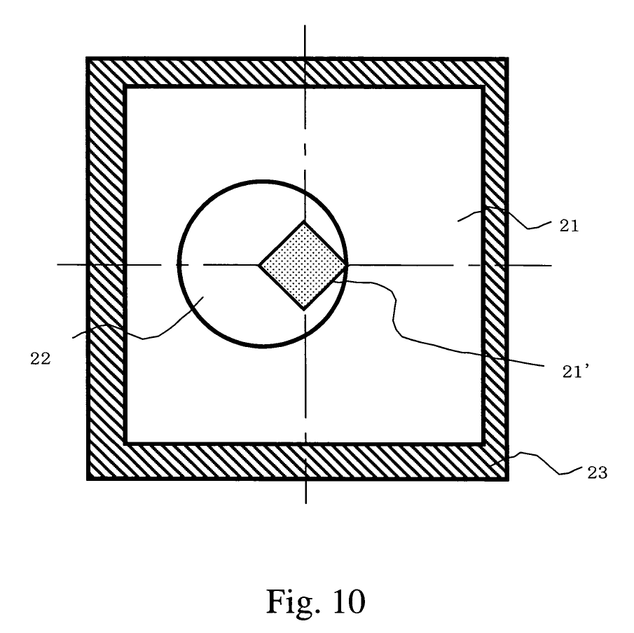

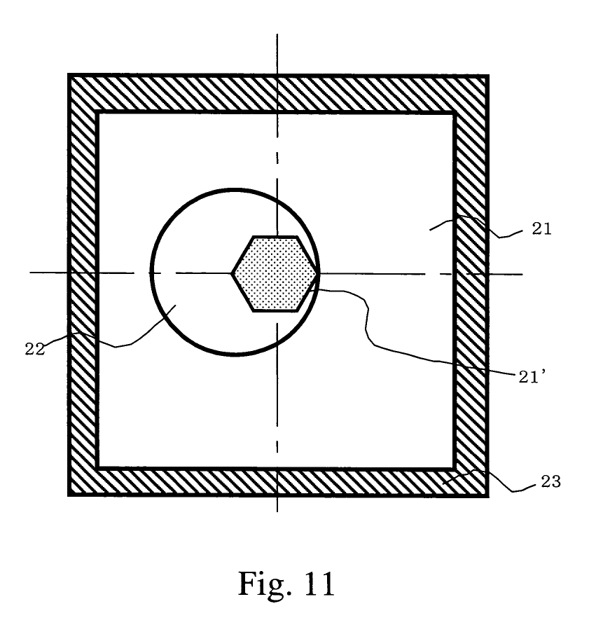

The plates and the probe-attached area of the embodiment may take a variety of configurations, with the only limitations being (1) that the holding plate is bigger enough to cover the moving plate during its movement, and (2) that the moving plate is bigger enough to cover the probe-attached area during its movement. In many embodiments, plates and the probe-attached area are assumed to be a circular, square (FIG. 10), rectangular, hexagon (FIG. 11), or any multi-side symmetrical geometric shape. For example, if the revolving plate is circular and with one probe-attached polygonal area, the diameter of the revolving plate is equal to or more than twice of a diagonal length of the probe-attached area. As another example, if the revolving plate and the probe-attached area are circular, the diameter of the moving plate R is equal to or more than twice of a diameter of the probe-attached area r, R≦2r, such as R=4 cm and r=2 cm, while the substrate is rectangular shaped. R≧2r in conjunction with limiting the amplitude of the relative movement ensures the edge of the moving plate do not move over the top of the edge of the probe-attached area. As such, there are always a safe margin therebetween such that no spillage of the reaction solution form the edges.

The holding plate may be manufactured from a variety of materials, while the moving plate is manufactured in combination of way(s) or material(s) to be hydrophilic. The plates may be manufactured from different materials as long as there is no interfere with the hybridization reagents or process. Specifically, the moving plate 2 can be made from glass or metal with an oxidization film formed on the surface entertaining the reaction solution. For example, the surface is processed with an amino-ethyl-amino-propyl-trimethoxycsilane aqueous solution with an acetic acid as a catalyst, and is left at room temperature for about 30 minutes. After washed with water, the surface is heated in air at about 110° C. for about one hour to introduce amino group therein. Thereafter, the surface is processed with acetic anhydride in an ethanol solvent at about 50° C. for about 30 minutes to displace its amino group with a carboxyl group. By means of coating the surface of the moving plate with carboxyl, the surface has negative charges in the water, i.e. hydrophilicity. In addition, it avoid any negative-charged substances, such as DNAs, attaches to the surface of the moving plate.

Generally, the surfaces of the plates and the probe-attached areas are typically substantially planar and smooth. However, in another embodiment, the surfaces may be replaced with another type to better suit different array shapes and sizes. Alternatively, the surfaces may be replaced with another type of surface having micro-channels or grooves therein to provide even better mixing effect.

### Embodiment 2

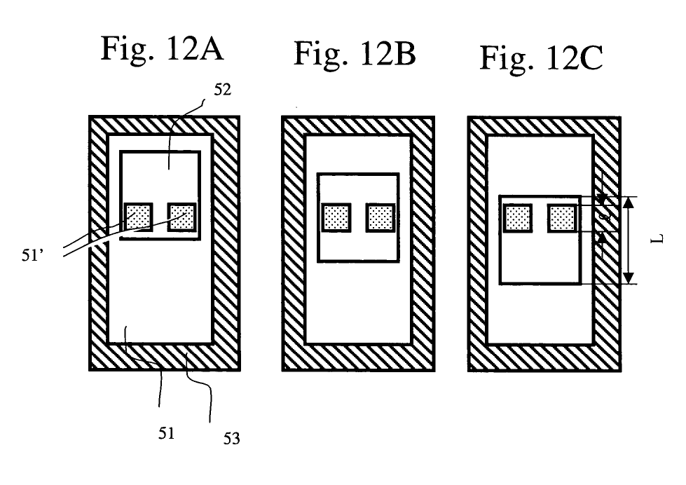

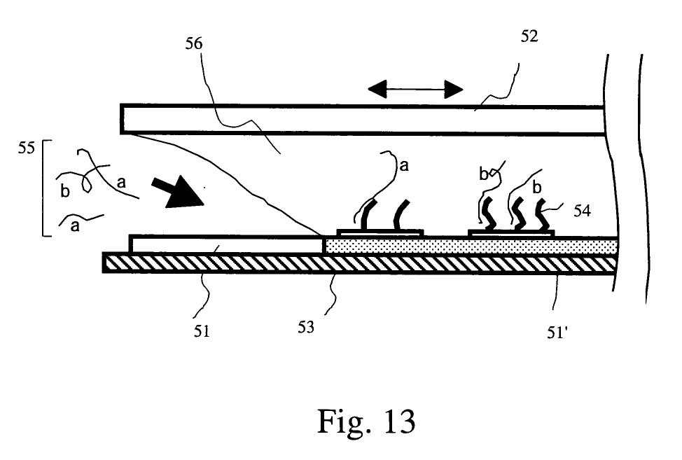

The second embodiment of the invention has a holding plate 53 holding a substrate 51 and a moving plate 52 move relatively to each other as shown in FIGS. 12A-12C to mix chemicals held on the surface (i.e., at least one substance of interest 25) of the substrate 51 with a chemical solution 56 accommodated therebetween with a gap from the chemicals as shown in FIG. 13. In particular, a pair of DNA probe-attached areas 51′ are held on the substrate 51, and the moving plate 22 moves in one direction forwards and backwards. Rather than revolving as the moving plate 2 of the first embodiment, the corresponding moving plate 52 in this embodiment moves in one direction back and forth.

The pair of probe-attached areas 51′ are attached with a plurality of probes 4 each of which captures a plurality of the substance of interest 55 as shown in FIG. 2. In another embodiment, two kinds of substances of interest 55a, 55b are attached to the DNA probe-attached areas 51′ respectively. Therefore, more than one kind of reactions can be conducted in the same space. (The number of the probe-attached areas is selected as two to simplify the discussion. Usually several probe-attached areas are used such that several different reactions are held in the same space)

The chemical solution 56 can be injected into the space between the substrate and the moving plate 52 with an solution injector 11 as described for the first embodiment. The plates and the probe-attached areas of this embodiment may take a variety of configurations, with the only limitations being (1) that the holding plate is bigger enough to cover the moving plate during its movement, and (2) that the moving plate is bigger enough to cover all the probe-attached areas during its movement. In many embodiments, plates and the probe-attached areas are assumed to be a circular, square or rectangular shape. For example, in the embodiment 2, the probe-attached areas and the moving plate are in a square shape with side lengths L≦21, such as L=48 nm and l=24 nm, while the holding plate is rectangular shaped. L≧21 in conjunction with limiting the amplitude of the relative movement ensures the edges of the moving plate do not move over the top of the edges of the probe-attached areas. As such, there are always safe margins therebetween such that no spillage of the reaction solution form the edges.

The moving speed (back or forth) of the moving plate is also 0.1-60 seconds per cycle as in the first embodiment. The preferred range is 0.9-30 seconds per cycle.

Sufficient fluorescence intensity was also obtained in reaction time in about 10 minutes according to the second embodiment. On the other hand, the set-still situation with dropping the hybridization solution onto the substrate reached only half of the fluorescence intensity after 180 minutes. That is, it was shown by both-way churning that a hybridization reaction is performed in a short time.

Moreover, the gap between the substrate and the moving plate that reaction efficiency increases along with the decrease of the gap (from 50 to 400 micrometers, preferably 50 to 100 micrometers) between the substrate and the moving plate like a case of the first embodiment.

Furthermore, the relative standard deviation of the fluorescence intensity of the spots in the probe-attached area drops to 1/11 by applying the solution mixing method according to the present invention. Thus, the invention significantly improve the homogeneity of hybridization.

The other aspects of the embodiment 2 are the same as those of the embodiment 1 such that the relevant description is omitted to avoid repetition.

### Embodiment 3

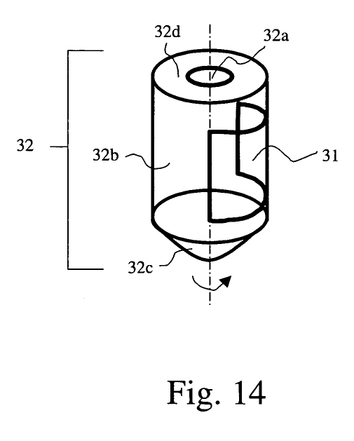

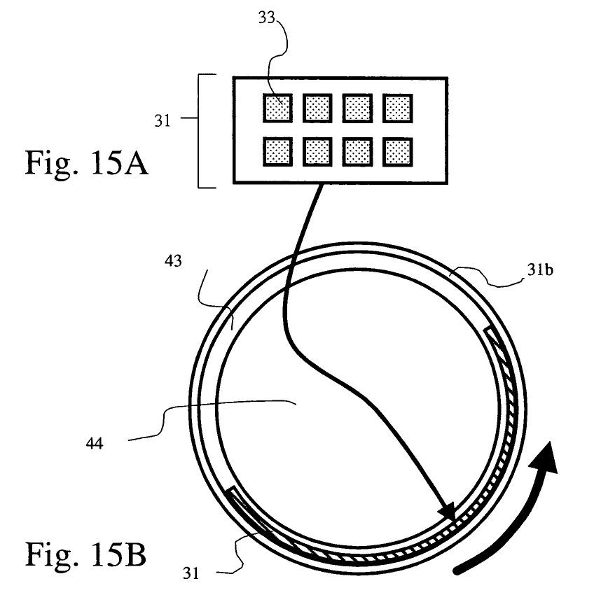

The third embodiment of the invention shown in FIG. 14 includes (1) a flexible substrate 31 having probe-attached areas, and (2) a spinning compartment 32 spinning around the central axis 32a. The spinning compartment mixes the chamial solution with the following steps: (i) setting the flexible substrate inside it by having the other surface from the surface with a probe-attached area face to an inner surface 32b of the spinning compartment 32, and (ii) keeping the chamical solution on the inner surface 32b by arising centrifugal force. This invention also equips with spinning means to have the spinning compartment spin around its central axis. The spinning means have some elements for spinning the spinning compartment, such as a spindle motor. A cover 32d is placed upon the spinning compartment 32 after the chemical solution and the flexible substrtate 31 (FIG. 15A) are placed inside the upon the spinning compartment 32.

FIG. 15B shows partial cross-sectional view of the spinning compartment in spinning. When the spinning compartment is spinning, the flexible substrate 31 faces to the inner surface 32b of the spinning compartment 32 with the other surface from the surface having the probe-attached area. Also, the flexible substrate 31 is forced to be pressed toward the inner surface 32b. FIG. 15A shows theflexible substrate 31 seen from the inside 44 of the spinning compartment 32. The chemical solution 43 set at the bottom 32c of the spinning compartment 32 is spread and covers onto the flexible substrate 31.

Faster spin speeds can result in stronger relative moving vectors of the flow of the solution, thus better the reaction efficiency. However, too high a spin speed can damage the probe-attached areas. The spinning speed is between 100 to 1000 rpm, and preferably 200 to 400 rpm.

The conventional rotisserie type rotating device/roller rolls along a horizontal axis (rather than a vertical axis) such that the array is soaked in the chemical solution only during a period of a rolling cycle (rather than during the whole cycle uniformly).

The flexible substrate and the spinning compartment of the embodiment may take a variety of configurations, with the only limitation being that the flexible substrate is smaller enough to be accommodated by the inner surface of the spinning compartment. In many embodiments, the substrate and the probe-attached areas are assumed to be a circular, square, rectangular, hexagon, or any multi-side symmetrical geometric shape. For example, in the embodiment 3, the probe-attached areas are square-shaped, while the holding plate is rectangular shaped.

Sufficient fluorescence intensity was obtained in reaction time in about 30 minutes according to the third embodiment. On the other hand, the set-still situation with dropping the hybridization solution onto the substrate reached only half of the fluorescence intensity after 180 minutes. That is, it was shown by spinning with the centrifugal force that a hybridization reaction is performed in a short time.

Furthermore, the relative standard deviation of the fluorescence intensity of the spots in the probe-attached area drops to ⅙ by applying the solution mixing method according to the present invention. In other words, the invention significantly improve the homogeneity of hybridization.

The other aspects of the Embodiment 3 are the same as those of the Embodiment 1 such that the relevant description is omitted to avoid repetition.

The top-down positions of the spining conpartment and the flexible substrate can be reversed. There are many conceivable variations of the dimensions of the described plates, probe-attached areas, spinning compartment as long as their structures are adapted to the respective requirements of the embodiments of the invention. The semiconductor structure does not have to be web-shaped, but rather can be adapted to the respective requirements.

The principles, preferred embodiments and modes of operation of the present invention have been described in the foregoing specification. However, the invention which is intended to be protected is not limited to the particular embodiments disclosed. The embodiments described herein are illustrative rather than restrictive. Variations and changes may be made by one skilled in the art without departing from the spirit of the present invention. Accordingly, it is expressly intended that all such variations or changes which fall within the spirit and scope of the present invention as defined in the claims, be embraced thereby.

What is claimed is:

1. A solution mixing apparatus for providing reactions therein, comprising:
a holding plate for holding at least one substrate on which at least one probe is immobilized; and
a moving plate with one surface being placed close to the substrate with a gap therein so as to accommodate a solution containing at least one substance therebetween, said moving plate moves relatively to the holding plate to mix the probe with the solution.

2. The solution mixing apparatus according to claim 1, wherein the moving plate moves relatively to the holding plate in one direction forwards and backwards.

3. The solution mixing apparatus according to claim 1, wherein the moving plate revolves relatively to the holding plate.

4. The solution mixing apparatus according to claim 1, wherein the reactions include bio-chemical reactions.

5. The solution mixing apparatus according to claim 1, wherein the gap ranges from 50,000-100,000 nm.

6. The solution mixing apparatus according to claim 1, wherein the moving plate is hydrophilic on the surface.

7. The solution mixing apparatus according to claim 1, wherein the apparatus is equipped with a temperature control apparatus.

8. The solution mixing apparatus according to claim 1, further comprising a solution injector for injecting the solution from an edge of a space between the substrate and the moving plate.

9. The solution mixing apparatus according to claim 1, further comprising a solution injector for injection the solution via a channel connecting through the moving plate to a space between the substrate and the moving plate.

10. The solution mixing apparatus according to claim 1, wherein the moving plate moves repeatedly in cycle and at a velocity of 0.1-60 second per one moving cycle.

11. The solution mixing apparatus according to claim 1, wherein the moving plate moves repeatedly in cycle and at a velocity of 0.9-30 second per one moving cycle.

12. The solution mixing apparatus according to claim 1, wherein a length of the moving plate along a direction the moving plate moves is equal to or more than twice of a length of an area where the probe is immobilized in the direction the moving plate moves.

13. The solution mixing apparatus according to claim 3, wherein the moving plate revolves about a probe-attached area on the substrate repeatedly in cycle like a hulahoop.

14. The solution mixing apparatus according to claim 3, wherein the revolving plate is circular and an area where the probe is immobilized is polygonal and a diameter of the revolving plate is equal to or more than twice of a diagonal length of the probe-immobilized area.

15. The solution mixing apparatus according to claim 3, wherein the moving plate and an area where the probe is immobilized are circular, and a diameter of the moving plate is equal to or more than twice of a diameter of the probe-immobilized area.

16. The solution mixing apparatus according to claim 3, further comprising a moving means which includes a base plate, at least one shaft, and at least one auxiliary plate, wherein the base plate has the moving plate fixed on one side and the auxiliary plate rotatably attached on another side, while the auxiliary plate has the base plate rotatably attached on one side and the shaft fixed on another side.

17. The solution mixing apparatus according to claim 16, wherein the moving means includes a pair of the shafts, and a pair of the auxiliary plates,
the moving plate, the base plate and the auxiliary plates are circular, and
the moving plate is fixed under a the base plate on one side of the base plate, a rotation axe of the auxiliary plate is a center of the shaft, and one shaft is fixed at a distance from a center of the auxiliary plate while another shaft is fixed at the same distance from a center of another auxiliary plate.

18. A solution mixing apparatus for providing reactions therein, comprising:
a compartment for setting a substrate attached with at least one probe on one surface onto an inner surface of the compartment with another surface to accommodate a solution thereon; and
spinning means for spinning the compartment about the central axis thereby spreading evenly and accommodating the solution containing at lest one substance on one inner surface therein.

19. A solution mixing method for providing reactions therein, comprising:
holding at least one substrate on which at least one probe is immobilized on a holding plate; placing a moving plate with one surface close to the surface of the holding plate thereby accommodating a solution including at least substance of interest therebetween with a gap from the probe held on thesubstrate; and
moving the moving plate relatively to the holding plate thereby mixing the probe and the solution.

20. The solution mixing method according to claim 19, wherein the substrate includes at least one probe-attached area, and
the moving plate and the probe-attached area are configured with shapes which work in conjunction with the relative movement to provide an equal solution flow volume at each location on the probe-attached area.
